(19) United States
(12) Patent Application Publication
(10) Pub. No.: US 2004/0030399 A1
(43) Pub. Date: Feb. 12, 2004

(54) Articulation prosthesis

(76) Inventor: Joseph-Guy Asencio, Nimes (FR)

Correspondence Address: YOUNG & THOMPSON, 745 SOUTH 23RD STREET 2ND FLOOR, ARLINGTON, VA, 22202

(21) Appl. No.: 10/296,160

(22) PCT Filed: May 17, 2001
(86) PCT No.: PCT/FR01/01509

(30) Foreign Application Priority Data
May 22, 2000 (FR) 00/06492

(51) Int. Cl. A61F 2/42; A61F 2/46

(52) U.S. Cl. 623/21.18

(57) ABSTRACT

A prosthesis, in particular for the ankle, has two nesting elements (2,4) assigned to a respective bone (12,22) and an intermediate element (6) interposed between them for relative articulation. Instruments for implanting the prosthesis are provided. The tibial element (2) comprises a centring brace (10) provided with elements to be gripped by the practitioner for extraction, consisting of a bend (24) in the brace (10) and by facets (26,28) provided at its distal end. The ankle bone element (4) comprises a plane (44) for longitudinal support against the ankle bone (22). A specific instrument enables to centre the ankle bone element (4) relative to the tibial element (2).

The invention is in the field of medical prostheses, and more particularly articulation prostheses between two bones, prosthesis of the ankle interposed between the tibia and the anklebone in particular. The invention also has for its object instruments for the implantation of such a prosthesis.

There are known ankle prostheses comprising a tibial element and an anklebone element, between which is interposed an intermediate element, movable relative to the anklebone element and stationary relative to the tibial element. The tibial element is constituted by an elongated shouldered member. The elongated portion, called a brace, is of overall conical configuration to penetrate axially within a recess in the tibia made by the practitioner, for centering the tibial element. The shoulder is constituted by a plate on which the tibia rests, the plate itself resting on the intermediate element. It will be noted that a front window is provided in the tibia to facilitate the emplacement of the tibial element. The anklebone element is constituted by a body of overall longitudinal cross-section shaped in a circular segment. The lower surface of the body is provided with a projecting member, such as a brace or rails, for nesting within an anklebone recess provided by the practitioner, for centering the anklebone element. The upper surface of the body is convex to support the lower correspondingly shaped surface of the intermediate element. The interposition of the intermediate element between the tibial and anklebone elements previously positioned on the corresponding bone, results in their compression with an axial force against the corresponding bone and because of this holding the assembly of the different elements of the prosthesis together.

A problem to be solved resides in the emplacement and positioning by the practitioner of the different elements on the bones. More particularly, the first difficulty resides in centering the elements within the bone relating thereto, relative to each other. It follows that the recesses provided extemporaneously must precisely correspond to the respective nesting members of the tibial and anklebone elements, failing which there can result a poor operation of the prosthesis.

Another problem to be solved resides in the fact that the use of elements of the prosthesis can be poorly adapted, and that a new procedure will be necessary for replacement of the elements initially emplaced. Finally, because of the shape itself of the anklebone, the centering member of the anklebone element cannot be extended inwardly of the bone following that of the tibial element. There results a general weakness of the prosthesis.

The object of the present invention is to provide an articulation prosthesis between two bones, particularly of the anklebone, permitting facilitating operations of preparation of the bones and for emplacing the elements of the prosthesis relating to them. Another object of the invention is to provide instruments for the preparation of the anklebone for the implantation of such a prosthesis. An another object of the invention is to facilitate the interchangeability between the different elements comprising the prosthesis. Finally, a last object of the invention is to improve the competitiveness of the prostheses of this type.

The articulation prosthesis between two bones of the present invention, particularly an ankle prosthesis, is of the type of prosthesis comprising two nesting elements provided to be introduced within a recess of a respective bone, made at the time by the practitioner, and an intermediate element interposed between the nesting elements for their articulation to each other and their compression between the corresponding bone. The nesting elements each comprise a distal member for positioning within the bone relating thereto and a proximal member for support against an opposed surface respectively of the intermediate element. The distal positioning element, the so-called brace, of one of the nesting elements, so-called tibial in the case of the example in which the corresponding bone is the tibia, is a centering member with an overall conical elongated shape narrowing toward its distal end, provided to penetrate axially within the corresponding- bone, particularly the tibia. The proximal support member for the tibial element against the corresponding surface of the intermediate member is itself shaped as a plate, connected to the proximal end of the brace with an overall orientation substantially perpendicular to the axis of extension of this latter.

According to a first aspect of the invention, the latter consists in providing the brace of the tibial element with means, so-called gripping, for gripping and extracting by the practitioner outside the recess which receives it. These means comprise the following characteristics, taken alone or in combination:

1) the brace comprises a medial inflection zone toward its distal end to form a surface, so-called holding surface, inclined relative to the external surface of general extension of the brace from its proximal end. These arrangements are such that the practitioner can take hold firmly on the holding surface by means of a tool, and exert a traction on the brace toward its proximal end for its extraction from the recess which receives it.

2) The holding surface comprises at least one overall flat configuration formed by at least one facet provided on the external surface of the brace. More particularly, this facet at least is provided on a portion of the external surface of the brace, so-called frontal, provided to be disposed facing the tibial window opened by the practitioner.

3) The connection face of the brace with the plate and the overall axis of extension of the brace, form together an angle less than 90°, comprised preferably between 80 and 88°. In other words, the brace comprises a general inclination relative to the plate, so as to increase the inclination of the holding surface relative to the overall orientation of this latter. It will be seen moreover that the inclination of the brace facilitates its swinging through the tibial window during emplacement or removal of the tibial element.

4) The brace comprises a truncated pyramidal configuration, of overall section which is triangular, trapezoidal or the like, and rounded edges. These arrangements are such that the brace is immobilized in rotation thanks to its non-circular cross-section, the centering of the tibial element taking place without risk of floating thanks to its positioning by means of lateral surfaces that are generally flat, of the brace.

According to a preferred embodiment, the connection means between the brace and the plate are nesting connection means, constituted by a coacting male member and female member, respectively provided on either one of the plate and the brace. These arrangements are such that the practitioner can, from a single plate, add to this latter a brace adapted to a specific use from a set of braces at his disposal. It results that the prosthesis of the invention is competitive because of the fact that the braces of different sizes can be produced independently of the plate, a same plate being adapted to be used for different braces.

According to a second aspect of the invention, the other nesting element of the prosthesis is of the type of element comprising an overall longitudinal cross-section shaped as a circular segment. Its positioning member is constituted by at least one rail, brace or the like, provided such as to be nested within the interior of a recess, such as a trench in correlation with the rail, provided in a corresponding bone, particularly the anklebone, for centering on this latter of the nesting element, so-called anklebone element, in the case of an example of a prosthesis of the ankle.

In a preferred embodiment of the knee bone element, the latter comprises at least one transverse support plane against longitudinal sliding of the anklebone element. This support plane is provided on the lower surface and to the rear of the anklebone element, and is preferably inclined by an angle C, so as to form a dihedral between the two planes, to limit the corresponding arrangements of the anklebone. As a result, the support of small extent of the anklebone element in the longitudinal direction which the nesting of the rail within the anklebone produces, is reinforced by its extended longitudinal support by means of the support plane which it comprises.

To prepare the surface of the anklebone provided to receive the longitudinal support plane of the anklebone element, there is provided a specific instrument comprising, at the end of a gripping sleeve, a plate provided with at least one positioning member on the anklebone and a window for the passage and guidance of a tool for preparing said surface.

It will be understood that the positioning member of the plate is preferably similar to that for positioning the anklebone element on the anklebone and is provided to be disposed within the corresponding recess previously produced by the practitioner. It results that the preparation of the longitudinal support surface of the anklebone element takes place in identical correspondence with the shape of this latter as to its centering on the anklebone.

The window for the passage and guidance of the tool is for example provided through a return of the plate, and is advantageously inclined relative to the rest surface of the anklebone by an angle D corresponding to angle C of inclination of the longitudinal support plane of the anklebone element on the anklebone. Preferably of oblong shape, its length is of the order of the width of the anklebone to be prepared.

According to another aspect of the invention, there is proposed an instrument for the centering of the tibial and anklebone elements relative to each other. This instrument, in known manner, comprises at the end of a gripping sleeve a plate provided with at least one window for the passage of a tool, adapted to make in the corresponding bone the recess provided to receive the positioning member of the anklebone element. According to the invention, the plate is provided moreover with a positioning member provided to coact with a supplemental member provided within the plate of a tibial element, particularly for test purposes, previously centered on the corresponding bone. It results that the positioning of the recess provided to receive the centering element of the anklebone element is made correlatively to the centering of the tibial element within the corresponding bone. It will be seen that according to this aspect of the invention, the tibial element comprises at its base a positioning member for a plate taking part in a corresponding instrument of the invention. It will also be understood that the positioning of the opening at least relative to the tibial element, is as much a transverse positioning relative to the axis of extension of the brace, as a positioning of its orientation relative to the anklebone.

For example, the positioning member of the plate is constituted by a lug provided to penetrate an opening provided in the base of the plate of the tibial element, the latter being preferably overall coaxial with the general axis of extension of the brace. The lug is itself positioned on the plate as a result.

According to a preferred embodiment, the plate comprises moreover at least one anchoring member within the corresponding bone, constituted for example by wedge to prevent untimely movement of the plate during the operation of preparing the recess provided to receive the positioning member of the anklebone element. It will be noted that in the case in which the positioning member of the plate relative to the tibial element is a figure of evolution, such as the above lug, the anchoring member is offset relative to the axis of extension of the rotatable member to permit holding the plate against pivoting.

Preferably, the plate and the positioning member of this latter on the tibial element are provided at the end of a respective branch of a grip, particularly with a rack, for spacing the bone from the articulation.

It will be understood that the arrangements foreseen relative to the centering element of the tibial and anklebone elements, one relating to the case of the example of an ankle prosthesis, are applicable on the one hand to any articulation prosthesis comprised by elements provided to be connected to a respective bone, and on the other hand a prosthesis of the ankle independently of the above characteristics of the invention, relative to each of these elements.

The present invention will be better understood from the details that will appear from the description which will be given for a preferred embodiment, with respect to the accompanying sheets of drawings, in which:

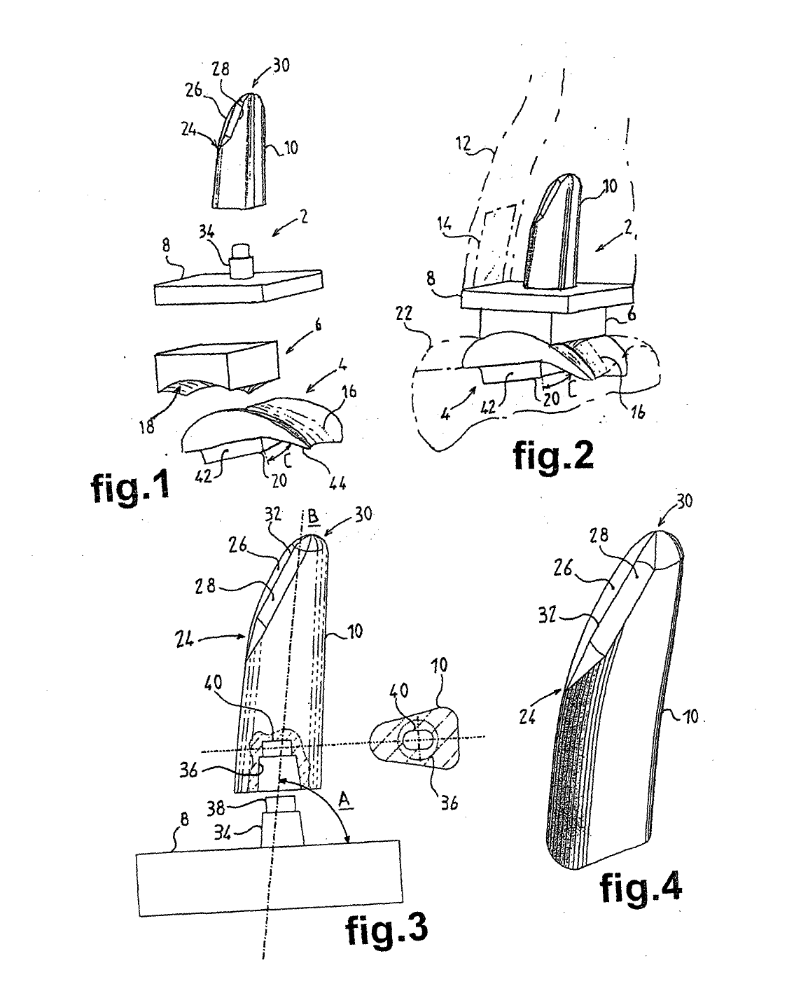

FIG. 1 and FIG. 2 are perspective illustrations of a prosthesis according to a preferred embodiment of the invention, respectively in an exploded view and in an assembled view.

FIG. 3 is an exploded cross-sectional view of a tibial element forming part of a prosthesis shown in FIGS. 1 and 2.

FIG. 4 is a perspective view of a brace forming part of a tibial element shown in FIG. 3.

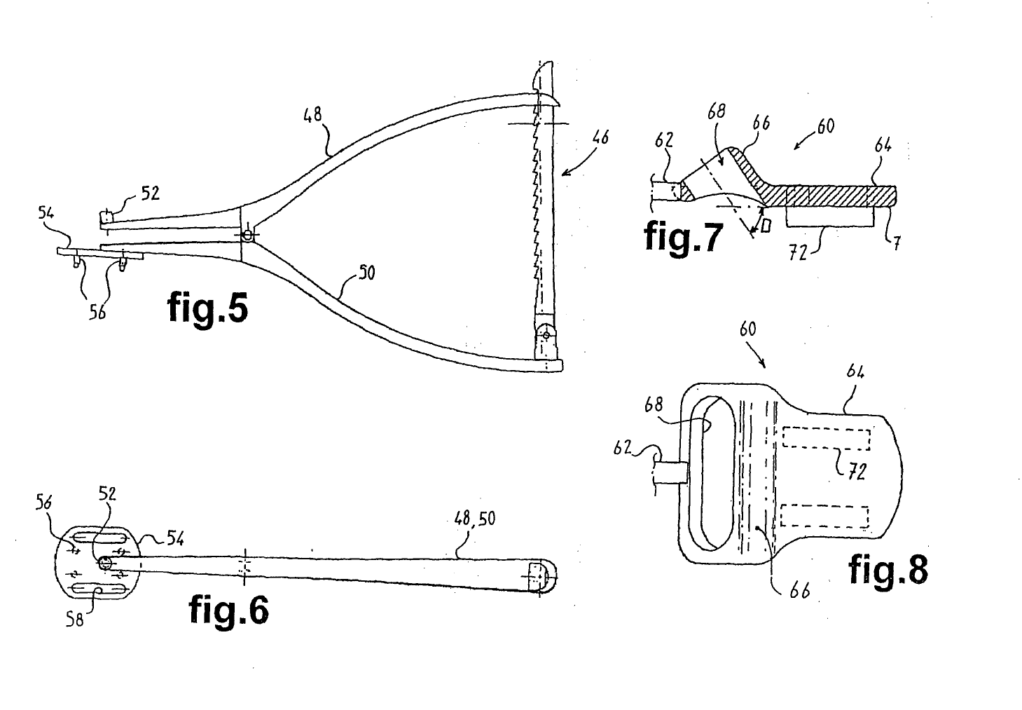

FIGS. 5 and 6 are illustrates respectively from the side and from above, of an instrument for centering the tibial and anklebone elements relative to each other, according to a preferred embodiment,

FIGS. 7 and 8 are fragmentary illustrations respectively in cross-section and from above, of an instrument for the preparation of the surface of the anklebone provided for longitudinal support of the anklebone element, according to a preferred embodiment.

In FIGS. 1 and 2, a knee prosthesis is comprised by a tibial element 2 and an anklebone element 4 between which is interposed an intermediate element 6 for their relative articlation.

The tibial element 2 comprises a plate 8 located at its proximal end, provided to rest on the upper surface of the intermediate 6. It moreover comprises a brace 10 at its distal end, provided to be introduced within a recess in the tibia 12 provided at the time by the practitioner, for centering the tibial element 2. There will be noted the presence of a front window 14 provided in the tibia 12 to facilitate the emplacement of the tibial element 2.

The anklebone element 4 is of a longitudinal cross-section overall shaped as a circular segment, and comprises itself at its proximal end an upper surface 16 longitudinally convex provided to coact with a lower concave surface 18 of the intermediate element 6. It will be noted that these contact surfaces 16 and 18, between the anklebone element 4 and the intermediate element 6, rae also transversely incurved for their relative transverse positioning.

In FIGS. 1 to 4, and according to a first aspect of the invention, the brace 10 of the tibial element 2 comprises a medial inflection zone 24 toward its distal end. This inflection 24 forms a surface 26, 28 for gripping the brace 10 for the extraction of the tibial element 2. This gripping surface 26, 28 is inclined toward the plate 8 relative to the external surface of general extension of the brace 10. Moreover, the general axis B of extension of the brace 10 is itself inclined relative to the general orientation of the plate, at an angle A of the order of 85°.

Referring more particularly to FIG. 4, the gripping surface 26, 28 is shaped in at least one flat of the external surface of the brace 10. More particularly, the gripping surface 26, 28 extends over the inflection region 24 of the brace 10, which is to say from its medial region to its distal end 30, or summit. The gripping surface 26, 28 is comprised by a plurality of facets provided on the external surface of the brace 10, of which at least one front facet 26 is next to two lateral facets 28.

It will be understood that by front facet 26 is meant a facet provided on the brace 10 in its region provided to be facing the tibial window 14 that has been made by the practitioner. It will be noted that the brace 10 comprises an overall triangular section with rounded edges. In the illustrated example, the front facet 26 extends over the front surface of the brace 10, and the lateral facets 28 extend from their proximal end toward their distal end, over a respective front surface and lateral surface of the brace 10, without however their transverse extension exceeding half the lateral surfaces of the brace 10. It will be thus noticed that preferably, the common edges, such as 32, between those of the lateral facets 28 and those of the front facets 26, have a converging orientation and tend to join at each of the ends of the facets 26, 28.

In other words, the front facets 26 and lateral facets 28 are tapered at their distal and proximal ends, to join respectively in the medial zone of the brace 10 and at the summit 30 of this latter.

In FIG. 3, the brace 10 and the plate 8 are connected to each other by means of a nesting cone 34 which comprises the plate 8, provided to penetrate in direct contact within the interior of a corresponding recess 36 of the brace 10. It will be noted that this nesting is completed by a non-circular nesting between a male member 38 of the plate 8 and a corresponding recess 40 of the brace 10, to immobilize this latter in rotation relative to the plate 8.

Returning to FIGS. 1 and 2, the anklebone 22 is at the time prepared by the practitioner, such that its dome can receive the base 20 of the anklebone element 4. Grooves are at the time provided in the anklebone 22 to receive the rails 42 for centering the anklebone element 4. This latter comprises moreover an inclined surface 44 forming a longitudinal support plane for the anklebone element on the anklebone. It will be understood that this inclination C is relative to the base surface 20 of the anklebone element.

In FIGS. 5 and 6, a clamp with a rack 46 is used to space the tibia 12 from the anklebone 22 for implantation of the prosthesis. The branches 48 and 50 of the clamp 46 comprise at the distal end a lug 52 on one (48) and a plate 54 on the other (50). The lug 52 is provided to be disposed in a corresponding recess of a tibial test element, whilst the plate 54 is provided to be anchored within the anklebone by means of lugs 56 with which it is provided. Thanks to these arrangements, the plate 54 is centered on the anklebone relative to the tibial element 2, and the practitioner can provide in the anklebone 22 the grooves to receive the rails 42 of the anklebone element 4. It will be noted through the plate 54 the presence of openings 58 for the passage of the tool conventionally used to make the grooves in the anklebone 22.

In FIGS. 7 and 8, an instrument 60 is used to prepare the rear surface of the anklebone 22, against which the anklebone element 4 bears longitudinally. This instrument comprises a sleeve 62 at the end of which is supported a plate 54 provided with a return 66. For centering on the anklebone 22, the plate 64 comprises rails 72 similar to those of the anklebone element 4, which are provided to be disposed within grooves relating thereto previously provided by the practitioner in the anklebone 22. The return 66 comprises itself a window 68 for the passage of a tool for preparation of the rear surface of the anklebone 22. It will be noted that the window 68 is inclined relative to the base 70 of the plate 60 provided to rest on the anklebone 22, by an angle D corresponding to the angle C of inclination of the longitudinal bearing plane 44 of the anklebone element 4.

1. Articulation prosthesis between two bones (12, 22), particularly an ankle prosthesis, of the type of prosthesis comprising two nesting elements (2, 4) provided to be introduced within a recess in a respective bone (12, 22), and an intermediate element (6) interposed between the nesting elements for their articulation to each other and their placing in compression against the corresponding bone, the nesting elements each comprising a distal member (10, 42) for positioning within an associated bone and a proximal member (8, 16) for bearing against an opposite surface respectively of the intermediate element (6), the distal positioning member (10), called a brace, of one of the nesting element (2), called a tibial element, being a centering member of an overall conical elongated shape narrowing toward its distal end, provided to penetration axially within the corresponding bone (12), particularly the tibia, the proximal bearing element (8) of the tibial element (2) against the corresponding surface of the intermediate member (6) being itself shaped as a plate connected to the proximal end of the brace with a general orientation substantially perpendicular to the axis B of extension of this latter (10), characterized:
in that the brace (10) comprises means (24, 26, 28, A), called gripping means, for its gripping and extraction by the practitioner from the recess which receives it, said means (24, 26, 28, A) comprising at least one zone (24) of inflection intermediate the brace (10) toward its distal end to form a so-called gripping surface (24, 26), inclined relative to the external surface of general extension of the brace (10) from its proximal end,
such that the practitioner can take firm hold against the gripping surface (24, 26) by means of a tool, and exert a traction on the brace (10) toward its proximal end for its extraction outside the recess that receives it.

2. Prosthesis according to claim 1, characterized:
in that the gripping means (24, 26, 28, A) of the brace comprise at least one overall flat formation on the gripping surface (26, 28), which is formed by at least one facet (24, 26) provided on the external surface of the brace (10).

3. Prosthesis according to any one of the preceding claims, characterized:
in that the gripping means (24, 26, 28, A) of the brace (10) comprise a general inclination A of the brace 10 relative to the plate (8), so as to increase the inclination of the gripping surface (26, 28) relative to the general orientation of this latter (8).

4. Prosthesis according to any one of the preceding claims, characterized:
in that the gripping means (24, 26, 28, A) of the brace (10) comprise a truncated pyramidal configuration of the brace (10), with an overall triangular cross-section with rounded edges.

5. Prosthesis according to claims 2 and 4, characterized:
in that the gripping surface is comprised by a plurality of facets (26, 28) provided on the external surface of the brace (10), of which at least one frontal surface (26) next to lateral facets (28), the front facets (26) and lateral facets (28) being tapered at their distal and proximal ends, to rejoin respectively an intermediate region of the brace (10) and at the summit (30) of this latter (10).

6. Prosthesis according to any one of the preceding claims, characterized:
in that the connection means (34, 38, 36, 40) between the brace (10) and the plate (8) are nesting connection means constituted by a male member (34, 38) and a female member (36, 40) coacting respectively and provided on either one of the plate (8) and the brace (10),
such that the practitioner can, from a single plate (8), add to this latter (8) a brace (10) adapted to a specific use from a set of braces (10) at his disposal.

7. Prosthesis according to any one of the preceding claims, the other of the nesting elements (4), called the anklebone element, comprising an overall longitudinal section in the form of a circular segment, and a positioning member (42) for centering it on the corresponding bone (22), particularly the anklebone, characterized:
in that the anklebone element (4) comprises at least one transverse bearing plane (44) against longitudinal sliding, provided on the lower surface (20) and to the rear of the anklebone element (4),
such that the support of the anklebone element (4) in the longitudinal direction due to the centering member (42) within the corresponding bone (22), is reinforced by its longitudinal bearing extent by means of the support plane (44) which it comprises.

8. Prosthesis according to claim 7, characterized:
in that said support plane (44) is inclined at an angle C to limit the corresponding arrangements of the anklebone (22).

9. Instrument for the preparation of the surface of the bone (22) provided to receive the longitudinal support plane (44) of the anklebone element (4) forming a process according to any one of claims 7 and 8, characterized:
in that it comprises, at the end of a gripping sleeve (62), a plate (60) provided with at least one positioning member (72) on the anklebone (22) and a window (68) for the passage and guidance of a tool for preparing said surface.

10. Instrument according to claim 9, characterized:
in that the positioning member (72) for the plate (60) is similar to that (42) for positioning the anklebone element (4) on the anklebone (22), and is provided to be disposed within the corresponding recess previously made by the practitioner,
such that the preparation of the longitudinal support surface of the anklebone element (4) takes place in identical correspondence with the configuration of this latter with respect to its centering on the anklebone (22).

11. Instrument according to any one of claims 9 and 10, for the preparation of the surface of the bone (22) provided to receive the longitudinal bearing plane of the anklebone element (4) forming part of a prosthesis according to claim 8, characterized:
in that said window (68) is provided through a return (66) of the plate (60) and is inclined relative to its rest surface (70) on the anklebone (22) at an angle D corresponding to that C of inclination of the longitudinal support plane (44) of the anklebone element (4) on the anklebone (22).

12. Instrument for centering relative to each other tibial and anklebone elements (2, 4) forming part of a prosthesis according to any one of claims 1 to 8, of the type of instrument comprising an end of a gripping sleeve (48, 50) a plate (54) provided with at least one opening (58) for the passage of a tool, adapted to provide in the corresponding bone (22) the recess provided to receive the positioning member (42) of the anklebone element (4), characterized:
in that the plate (54) is provided with a positioning member (52) provided to coact with a supplemental member provided inside the plate (8) of the tibial element (2), previously centered on the corresponding bone (12),
such that the positioning of the recess provided to receive the centering member (42) of the anklebone element (4) is provided correlatively to the centering of the tibial element (2) within the corresponding bone (12).

13. Instrument according to claim 12, characterized:
in that the positioning member (52) of the plate (54) is constituted by a lug provided to penetrate an opening provided in the base of the plate (8) of the tibial element (2).

14. Instrument according to any one of claims 12 and 13, characterized:
in that the plate (54) comprises at least one anchoring member (56) within the corresponding bone (22) so as to prevent an unexpected movement of the plate (54) during the operation of preparing the recess to receive the positioning member (42) of the anklebone element (4).

15. Instrument according to claim 14, characterized:
in that the positioning member (52) of the plate (54) relative to the tibial element (2) being a member of rotation, said anchoring member (56) is offset relative to the axis of extension of said revolution member (52) to promote holding of the plate (54) against pivoting.

16. Instrument according to any one of claims 12 to 15, characterized:
in that the plate (54) and the positioning member (52) of this latter on the tibial element (2) are provided at th end of a respective branch (48, 50) of a clamp (46) for spacing the bones (12, 22) of the articulation.
